(19) United States
(12) Patent Application Publication
(10) Pub. No.: US 2012/0322083 A1
(43) Pub. Date: Dec. 20, 2012

(54) ANTIBODY BINDING TO ABCA1 POLYPEPTIDE

(75) Inventors: Barbara Ecabert, Therwil (CH); Hugues Matile, Basel (CH); Everson Nogoceke, Basel (CH); Bernhard Reis, Basel (CH); Haiyan Wang, Allschwil (CH)

(73) Assignee: Hoffmann-La Roche Inc., Nutley, NJ (US)

(21) Appl. No.: 13/517,863

(22) Filed: Jun. 14, 2012

(30) Foreign Application Priority Data
Jun. 15, 2011 (EP) 11169924.5
Jul. 20, 2011 (EP) 11174755.6

(51) Int. Cl. C07K 16/18; C12P 21/00; C12N 15/63; C12N 15/13; C12N 1/21; C12N 5/18; G01N 33/566

(52) U.S. Cl. 435/7.21; 435/332; 435/69.6; 530/389.8; 530/388.2; 536/23.53; 435/252.3; 435/320.1

(57) ABSTRACT

The invention relates to an isolated antibody that binds to native ABCA1 polypeptide and its uses for the detection of ABCA1 polypeptides in tissue samples.

### RELATED APPLICATIONS

This application claims the benefit of EP Application No. 11174755.6, filed Jul. 20, 2011 and EP Application No. 11169924.5, filed Jun. 15, 2011. All the teachings of the above-referenced applications are incorporated herein by reference.

### SEQUENCE LISTING

The instant application contains a Sequence Listing which has been submitted in ASCII format via EFS-Web and is hereby incorporated by reference in its entirety. Said ASCII copy, created on May 10, 2012, is named P4713SeqList.txt and is 20,220 bytes in size.

### FIELD OF THE INVENTION

The present invention relates to antibodies binding to ABCA1 polypeptide and its uses in methods to detect ABCA1 polypeptide.

### BACKGROUND

The ATP-binding cassette transporter Al (ABCA1) is an ATP dependent transporter mediating the efflux of cholesterol and phospholipids to extracellular lipid poor HDL particles. The amino acid sequence of human ABCA1 polypeptide is given in SEQ ID NO: 1. ABCA1 is essential for the assembly of nascent HDL particles by phospholipid and apolipoprotein. ABCA1 functions as a pivotal regulator of lipid efflux from cells to apolipoproteins and is thus involved in lowering the risk of atherosclerosis. ABCA1 is pivotal in influencing plasma HDL levels.

Active in liver and small intestine, generating most circulating HDL. Defects in the gene encoding for the ABCA1 were shown to be one of the genetic causes for familial hypoalphalipoproteinemia (FHA).

Commercially available anti ABCA1 antibodies are not suitable for the detection of native ABCA1 polypeptide in tissue samples.

Therefore, there is a need for anti ABCA1 antibodies capable of detecting native ABCA1 polypeptide in tissue samples.

### SUMMARY OF THE INVENTION

In a first aspect the present invention relates to an isolated antibody that binds to native ABCA1 polypeptide.

In a particular embodiment, the ABCA1 polypeptide is human ABCA1 polypeptide.

In a further particular embodiment, the anti-ABCA1 antibody is a monoclonal antibody.

In a further particular embodiment, the antibody has been produced by immunizing suitable animals with whole cells expressing the ABCA1 polypeptide, preferably human ABCA1 polypeptide.

In a further particular embodiment, the antibody comprises a CDR3 of a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111) and a CDR3 of a VL domain of an antibody obtainable from a hybridoma cell selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another particular embodiment, the antibody comprises CDR1 to CDR3 of a VH domain of an antibody obtainable from a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111) and a CDR1 to CDR3 of a VL domain of an antibody obtainable from a hybridoma cell selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In a further particular embodiment, the antibody comprises a VH domain and a VL domain of an antibody obtainable from hybridoma cell line selected from the group consisting of ABCA1-3/84 (DSM ACC3109), hybridoma cell line ABCA1-3/125 (DSM ACC3110) and hybridoma cell line ABCA1-4/18 (DSM ACC3111).

In a further particular embodiment, the antibody is produced by hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another aspect the present invention relates to a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another aspect the present invention relates to an isolated nucleic acid comprising a sequence encoding a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another aspect the present invention provides an isolated nucleic acid comprising a sequence encoding a VL domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another aspect the present invention provides an isolated nucleic acid comprising a sequence encoding an antibody produced by a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

In another aspect the present invention provides a vector comprising a nucleic acid of the present invention and a host cell comprising a vector of the present invention.

In another aspect the present invention provides a method of producing an antibody comprising culturing a host cell of the present invention so that the antibody is produced.

In another aspect the present invention provides a use of the antibody of the present invention for the detection of ABCA1 polypeptide in a tissue sample of an animal.

In a particular embodiment of the use of the present invention the tissue sample is whole blood.

In a particular embodiment of the use of the present invention the animal is a human subject.

In another aspect the present invention provides a method for the detection of ABCA1 polypeptide in a tissue sample of an animal comprising:
a) providing a tissue sample of the animal,
b) detecting ABCA1 polypeptide in the sample of step a) using the antibody of the present invention.

In a particular embodiment, the tissue sample is whole blood.

In a particular embodiment, the animal is a human subject.

In a particular embodiment the detection of ABCA1 polypeptide in step b) is done by Flow Cytometry.

### BRIEF DESCRIPTION OF THE FIGURES

The patent or application file contains at least one drawing executed in color. Copies of this patent or patent application publication with color drawing(s) will be provided by the U.S. Patent and Trademark Office upon request and payment of the necessary fee.

FIG. 1 is a visualization of the term “staining index”. Blue: FLP293 cell line expressing ABCAl; White: parental cell line 293.

FIG. 2a: Detection of ABCA1 surface expression in a FLP293 cell line expressing ABCA1 (FLP293/ABCA1 cell line) using the commercial anti-ABCA1 antibody Novus DyLight 488 in FACS assay; Staining index is 1.54 (fluorescence FLP293ABCA1 cell line /fluorescence FLP293 parental cell line); Blue: FLP293 cell line expressing ABCA1; White: parental cell line 293. Antibody dilution: 1:200

FIG. 2b: Detection of ABCA1 surface expression in a FLP293 cell line expressing ABCA1 (FLP293/ABCA1 cell line) using the commercial anti-ABCA1 antibody ab81950 biotin in FACS assay; Staining index is 1.24 (fluorescence FLP293ABCA1 cell line /fluorescence FLP293 parental cell line); Blue: FLP293 cell line expressing ABCA1; White: parental cell line 293. Antibody dilution: 1:200

FIG. 2c: Detection of ABCA1 surface expression in a FLP293 cell line expressing ABCA1 (FLP293/ABCA1 cell line) using the commercial anti-ABCA1 antibody Novus biotin in FACS assay; Staining index is 1.28 (fluorescence FLP293ABCA1 cell line/fluorescence FLP293 parental cell line); Blue: FLP293 cell line expressing ABCAl; White: parental cell line 293. Antibody dilution: 1:200

FIG. 2d: Detection of ABCA1 surface expression in a FLP293 cell line expressing ABCA1 (FLP293/ABCA1 cell line) using the commercial anti-ABCA1 antibody ab18180 in FACS assay; Staining index is 1.41 (fluorescence FLP293ABCA1 cell line/fluorescence FLP293 parental cell line); Blue: FLP293 cell line expressing ABCAl; White: parental cell line 293. Antibody dilution: 1:200

FIG. 2e: Detection of ABCA1 surface expression in a FLP293 cell line expressing ABCA1 (FLP293/ABCA1 cell line) using the commercial anti-ABCA1 antibody ab66217 in FACS assay; Staining index is 1.84 (fluorescence FLP293ABCA1 cell line/fluorescence FLP293 parental cell line); Blue: FLP293 cell line expressing ABCA1; White: parental cell line 293. Antibody dilution: 1:200

FIG. 3a: Detection of ABCA1 surface expression in a FLP293 cell line expressing ABCA1 (FLP293/ABCA1 cell line) using the inventive anti-ABCA1 antibody ABCA1-3/84 in FACS assay; Staining index is 8.9 (fluorescence FLP293ABCA1 cell line /fluorescence FLP293 parental cell line); Blue: FLP293 cell line expressing ABCAl; White: parental cell line 293. Antibody concentration: 0.1 μg/ml

FIG. 3b: Detection of ABCA1 surface expression in a FLP293 cell line expressing ABCA1 (FLP293/ABCA1 cell line) using the inventive anti-ABCA1 antibody ABCA1-3/84 in FACS assay; Staining index is 10.4 (fluorescence FLP293ABCA1 cell line/fluorescence FLP293 parental cell line); Blue: FLP293 cell line expressing ABCAl; White: parental cell line 293. Antibody concentration: 1 μg/ml

FIG. 3c: Detection of ABCA1 surface expression in a FLP293 cell line expressing ABCA1 (FLP293/ABCA1 cell line) using the inventive anti-ABCA1 antibody ABCA1-3/84 in FACS assay; Staining index is 10.5 (fluorescence FLP293ABCA1 cell line /fluorescence FLP293 parental cell line); Blue: FLP293 cell line expressing ABCA1; White: parental cell line 293. Antibody concentration: 10 μg/ml

FIG. 4a: Detection of ABCA1 surface expression in a FLP293 cell line expressing ABCA1 (FLP293/ABCA1 cell line) using the inventive anti-ABCA1 antibody ABCA1-3/125 in FACS assay; Staining index is 49.7 (fluorescence FLP293ABCA1 cell line /fluorescence FLP293 parental cell line); Blue: FLP293 cell line expressing ABCAl; White: parental cell line 293. Antibody concentration: 0.1 μg/ml

FIG. 4b: Detection of ABCA1 surface expression in a FLP293 cell line expressing ABCA1 (FLP293/ABCA1 cell line) using the inventive anti-ABCA1 antibody ABCA1-3/125 in FACS assay; Staining index is 47.0 (fluorescence FLP293ABCA1 cell line /fluorescence FLP293 parental cell line); Blue: FLP293 cell line expressing ABCAl; White: parental cell line 293. Antibody concentration: 1 μg/ml

FIG. 4c: Detection of ABCA1 surface expression in a FLP293 cell line expressing ABCA1 (FLP293/ABCA1 cell line) using the inventive anti-ABCA1 antibody ABCA1-3/125 in FACS assay; Staining index is 46.9 (fluorescence FLP293ABCA1 cell line /fluorescence FLP293 parental cell line); Blue: FLP293 cell line expressing ABCAl; White: parental cell line 293. Antibody concentration: 10 μg/ml.

FIG. 5: Upregulation of ABCA1 on CD14+ monocytes with two different LXR agonists and LPS; Whole blood was incubated for 24h with LXR agonists or LPS. Detection of ABCA1 was done by FACS using anti-ABCA1 antibody ABCA1-3/125.

FIG. 6: Western Blot analysis of generated anti-ABCA1 monoclonal antibodies ABCA1-3/84, ABCA1-3/125 and ABCA1-4/18. The following cell lysates were used in the Western Blot to test the specificity of the generated anti-ABCA1 monoclonal antibodies:

Lane 1: stimulated THP1 cell-Lysate,

Lane 2: non stimulated THP1 cell-Lysate,

Lane 3: FLP293-hu-ABCA1 (FLP293 cell line expressing ABCA1),

Lane 4: FLP293 (negative control),

Lane 5: MWM (Molecular Weight Marker),

Lane 6: HEK-293-hu-ABCAl(DB272-in pANITA2)-6His (HEK cell line expressing human ABCA1 protein with a His tag).

FIG. 7: Western blot analysis of ABCA1 protein. Clone 4 expresses a large amount of ABCA1 indicated by detection of the full-length protein (approx. 240-kDA) and several smaller species. In comparison, no ABCA1 protein was detected in the untransfected FLP cell line.

FIG. 8: qPCR analysis of ABCA1 mRNA expression: The Cp values for the housekeeping gene GAPDH remained constant between the ABCAl-overexpressing Clone 4 vs. the parental line (18.00+0.24 vs. 18.56+0.20). In contrast, the Cp values indicated extremely low expression of ABCA1 mRNA in the untransfected line (36.00+0.73) vs. high levels in the Clone4-ABCA1 line (21.64+0.48). Using the ddCp method to calculate the difference indicates that Clone 4 expresses 21,000 times more ABCA1 transcripts compared to the parental FLP cell line.

FIG. 9: Detection of ABCA1 surface expression in THP-1 cells using the inventive anti-ABCA1 antibody ABCA1-4/18 in a FACS assay. THP-1 cells were treated with LXR agonist overnight to induce ABCA1 expression or control treated with DMSO control. Cells were stained with 1 μg/ml ABCA1 specific antibody (clone 4/18) or isotype control followed by staining with secondary PE conjugated antibody against mouse IgG1. Anti-ABCA1 antibody ABCA1- 4/18 gives a good separation of basal ABCA1 signal from isotype control.

FIG. 10: Detection of ABCA1 surface expression in THP-1 cells using the inventive anti-ABCA1 antibody ABCA1-3/125 in a FACS assay. THP-1 cells were treated with LXR agonist overnight to induce ABCA1 expression or control treated with DMSO control. Cells were stained with 1 μg/ml ABCA1 specific antibody (clone 3/125) or isotype control followed by staining with secondary PE conjugated antibody against mouse IgG1.

FIG. 11: Human tissue distribution of ABCA1 proteins shown by Western Blot using mouse anti-ABCA1 mAb clone 3/84.

FIG. 12: Immunohistochemistry staining of ABCA1 in human liver with mouse anti-ABCA1 mAb clone 3/84. Green: ABCA1 protein localized in cell membranes of hepatocytes, and Blue: Nuclei DAPI stained.

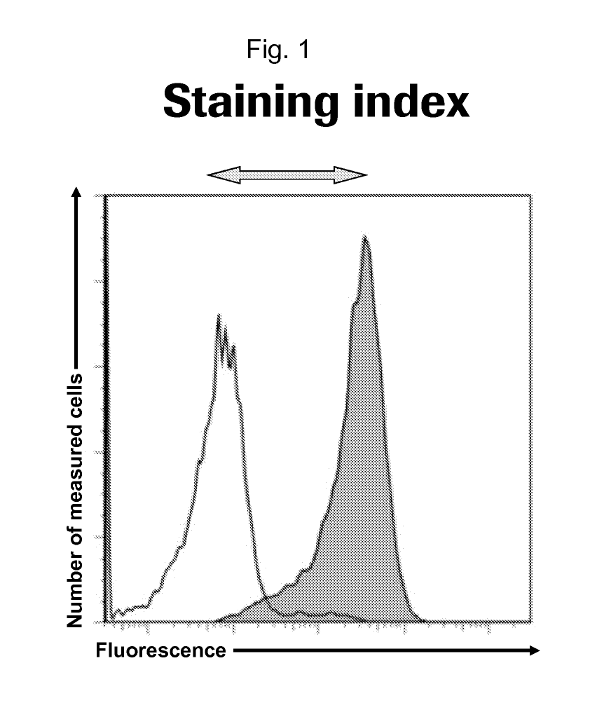

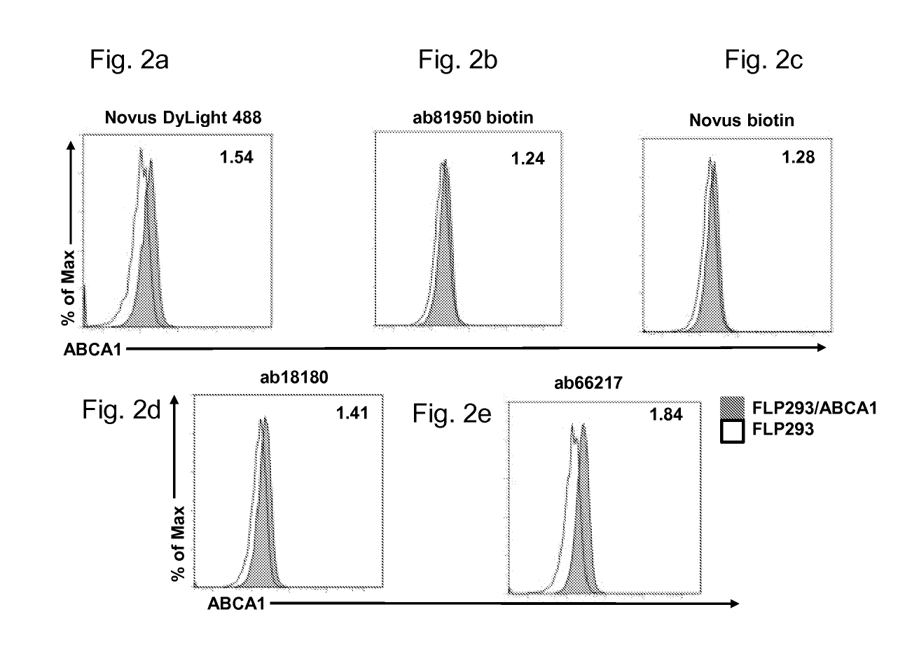

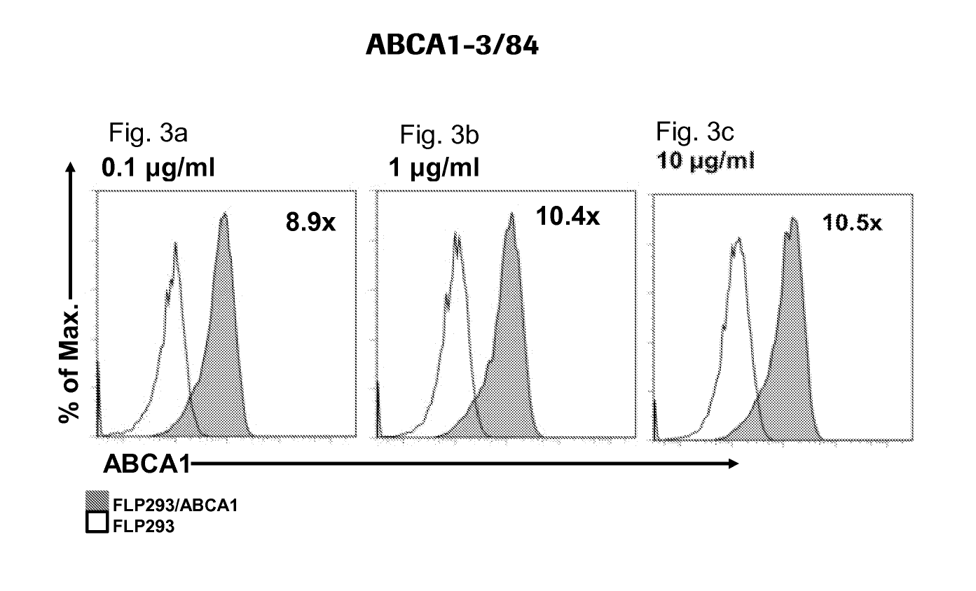

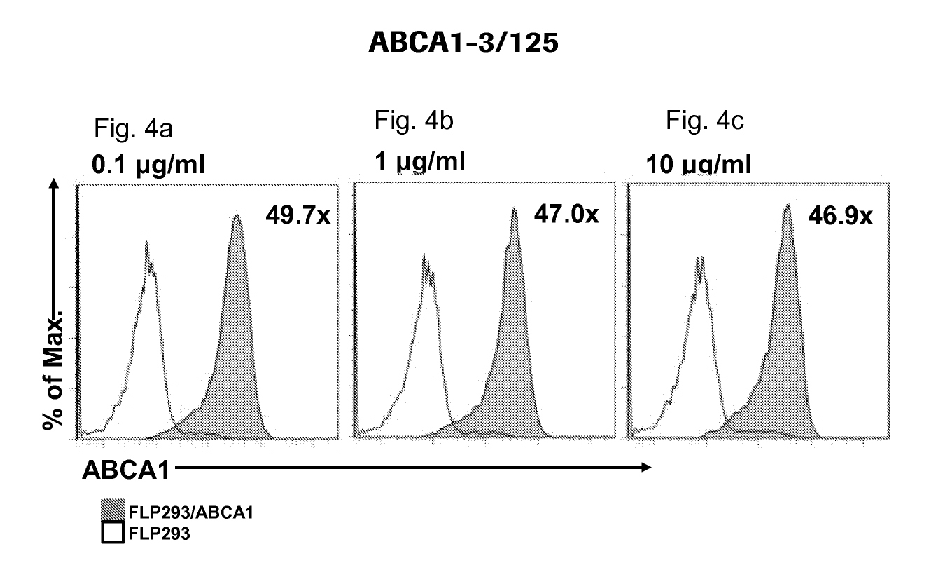

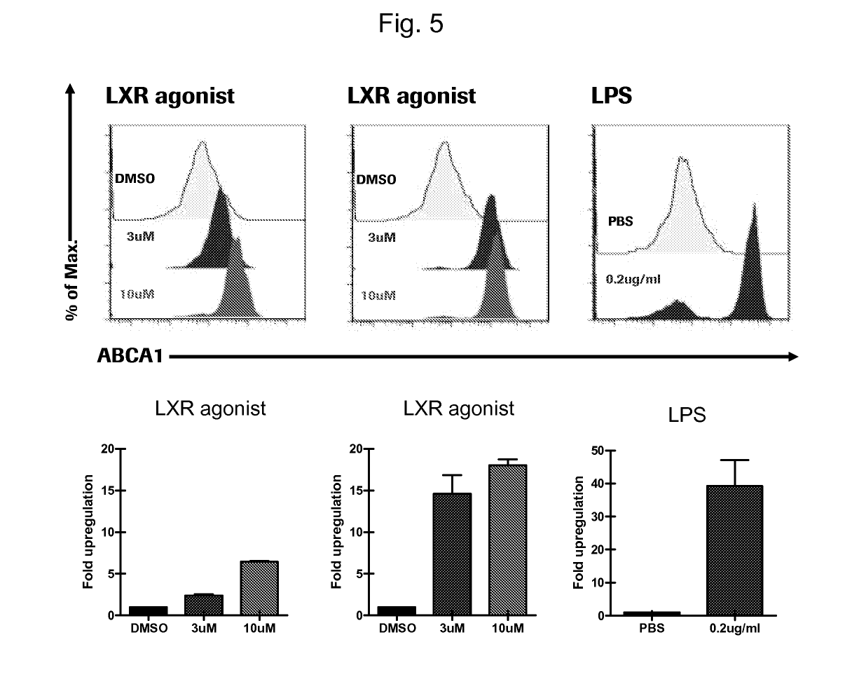

### DETAILED DESCRIPTION OF EMBODIMENTS OF THE INVENTION

The term “staining index” as used herein is defined as follows:

$$Staining index : \frac{ \begin{matrix}Median fluorescence intensity \\ ABCA 1 expressing cell line\end{matrix} }{\begin{matrix}Median fluorescence intensity \\ ABCA 1 \text{-} negative parental cell line\end{matrix}}$$

The term “antibody” encompasses the various forms of antibody structures including but not being limited to whole antibodies and antibody fragments. The antibody according to the invention can be a humanized antibody, chimeric antibody, or further genetically engineered antibody as long as the characteristic properties according to the invention are retained.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g. described in Houston, J. S., Methods in Enzymol. 203 (1991) 46-96). In addition, antibody fragments comprise single chain polypeptides having the characteristics of a V_{H} domain, namely being able to assemble together with a V_{L} domain, or of a V_{L} domain binding to ABCA1, namely being able to assemble together with a V_{H} domain to a functional antigen binding site and thereby providing the property.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J.D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation.).

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The “variable domain” (variable domain of a light chain (V_{L}), variable domain of a heavy chain (V_{H})) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The term “ABCA1 polypeptide” is used herein to refer to native ABCA1 polypeptide from any animal, e.g. mammalian species, including humans, and ABCA1 variants. The ABCA1 polypeptides may be isolated from a variety of sources, including human tissue types or prepared by recombinant and/or synthetic methods. The amino acid sequence of human ABCA1 polypeptide is given in Seq. Id. No. 1.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-ABCA1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Antibodies may be produced using recombinant methods and compositions, e.g., as described in U.S. Pat. No. 4,816,567. In one embodiment, isolated nucleic acid encoding an anti-ABCA1 antibody described herein is provided. Such nucleic acid may encode an amino acid sequence comprising the V_{L} and/or an amino acid sequence comprising the V_{H} of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-ABCA1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody of the present invention, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

Methods to clone antibody genes from hybridomas producing monoclonal antibodies are know to a person skilled in the art. For example, the genetic information for the variable heavy and light chain domains (V_{H} and V_{L}) can be amplified from hybridoma cells using polymerase chain reaction (PCR) with immunoglobulin-specific primers (Methods Mol Med. 2004; 94:447-58). The nucleic acid encoding the variable heavy and light chain domains (V_{H} and V_{L}) can then be cloned in a suitable vector for expression in host cells.

Methods for detection and/or measurement of polypeptides in biological samples are well known in the art and include, but are not limited to, Western-blotting, Flow cytometry, ELISAs or RIAs, or various proteomics techniques. For example, an antibody capable of binding to the denatured proteins, such as a polyclonal antibody, can be used to detect ABCA1 polypeptide in a Western Blot. An example for a method to measure a polypeptide is an ELISA. This type of protein quantitation is based on an antibody capable of capturing a specific antigen, and a second antibody capable of detecting the captured antigen.

A preferred method for the detection of native ABCA1 polypeptide is flow cytometry. Flow cytometry methods are described in Handbook of Flow Cytometry Method, J. Paul Robinson (Editor); Flow Cytometry—A Basic Introduction, Michael G Ormerod (2008) and Current Protocols in Cytometry (2010), Wiley.

### EXAMPLES AND METHODS

Monoclonal Anti Human ABCA1 Antibodies of the Present Invention

The following three mouse hybridoma cell lines producing monoclonal antibodies against human ABCA1 have been deposited with Deutsche Sammlung von Mikroorganismen and Zellkulturen GmbH (DSMZ), Mascheroder Weg lb, D-38124 Branuschweig, Germany, under the provisions of the Budapest Treaty on January 20, 2011 in the name of F. Hoffmann-La Roche Ltd. and received the below listed deposit numbers:
ABCA1-3/84=DSM ACC3109
ABCA1-3/125=DSM ACC3110
ABCA1-4/18=DSM ACC3111

Monoclonal Anti Human ABCA1 Antibody Generation

Expression of ABCA1 Protein on the Cell Surface of Mammalian Cells.

The human ABCA1 extracellular domain was expressed on the cell surface of HEK cells using the expression plasmid pANITA2. HEK-derived cell lines expressing human ABCA1 extracellular domain were established by stable transfection.

To obtain highly expressing cell lines, transfectants were separated into high-expressing cell-pools by fluorescent-activated-cell-sorting after surface staining with anti-FLAG antibodies. The mean fluorescence intensity of the cells gated for sorting into the high-expressing cell pool was 2.1-4.3 times higher than that of all transfectants.

Human ABCA1-expressing cell lines were tested for expression by Western blot analysis, showing a high level of expression of a protein with the expected molecular weight

Development of ABCA1 Specific Antibodies in Mice Immunised with Transfected HEK Cells

Our approach utilizes stably transfected mammalian cells (HEK293) expressing recombinant antigens on their cell surface. The transfected cells are also used for measuring seroconversion, hybridoma selection and antibody characterization. By presenting the antigen in its native conformation for immunization and hybridoma selection, this procedure promotes the generation of antibodies capable of binding to the endogenous protein.

The immunogen was obtained by immunopurifying the recombinant protein resulting in an immune complex and the use of these immune complexes as Immunizing Antigen. Immunepurification was confirmed by Western blot analysis using anti- His tag monoclonal antibodies.

Spleen cells of mice immunised with the immune complex were fused with PAI myeloma cells to generate B cell hybridoma. Fused cells were distributed in microtitre culture plate wells. To identify hybridoma cells that produce ABCA1 specific antibodies a two-step screening procedure was used that completely obviates the requirement for purified recombinant proteins. First all culture wells were tested for IgG production by ELISA. In a second step all wells positive for IgG production were screened for antibody binding to transfected cells by IFA. Transfected and non-transfected HEK cells spotted onto multiwell glass-slides were stained with individual hybridoma supernatants and analysed by fluorescence microscopy. Non-transfected HEK cells served as a negative control for each sample.

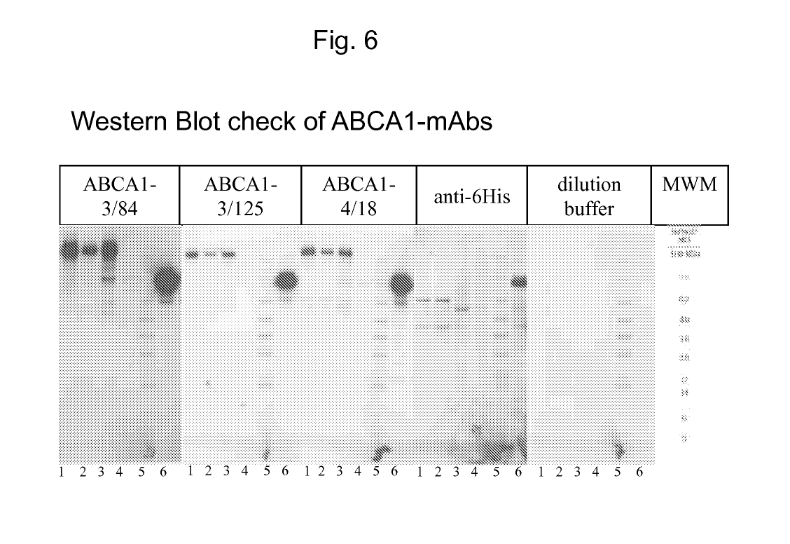

The specificity of generated monoclonal antibodies ABCA1-3/84, ABCA1-3/125 and ABCA1-4/18 was checked by Western Blot (FIG. 6). THP1=Human monocytic cell line.

Cell Culture

Human embryonic kidney cells (Flip-In 293 cells, Invitrogen), were maintained in Dulbecco's modified Eagle's medium (DMEM) supplemented with 10% fetal bovine serum, 2 mM L-glutamine, and antibiotics. Cells were transfected using Fugene-6 (Roche Biochemicals) as described by the manufacturer.

Human peripheral blood monocyte cells (THP-1, ATCC F-6430) were cultured in RPMI-1640 Glutamax (Invitrogen), supplemented with 0.05 mM 2-Mercaptoethanol (Invitrogen) and 10% heat inactivated FBS (Invitrogen). Cell concentrations did not exceed 1E06 cells/ml. Maximum passage number was 30.

Plasmids: A 6.5-kb DNA fragment encoding full-length human ABCA1 was subcloned into XhoI digested, Klenow filled plasmid PN721. After sequence verification, the 6.5-kb fragment was recovered and further subcloned into plasmid pCDNA5-FRT (Invitrogen).

Generation of an ABCA1 Stable Cell Line

A monoclonal stable cell line expressing human ABCA1 was obtained by co-transfecting pcDNA5-FRT-ABCA1 and pOG44 plasmids together (Invitrogen) using Fugene-6 (Roche Biochemicals) in growth medium in the absence of zeocin according to the manufacturer's recommendations. One day following transfection, hygromycin B was added to a final concentration of 50 μg per ml, and media changed every 3-4 days until hygromycin B-resistant colonies appeared. Individual colonies were selected and further propagated in the presence of hygromycin B. Individual clones were evaluated for ABCA1 expression by quantitative PCR and Western blot analysis. Based on these results, Clone 4 was selected as a high ABCA1-expressing cell line based.

Western blot analysis of ABCA1 protein expression: FLP or Clone 4 cells were cultured at 10⁶ cells per well in 12-wells plate for 48 hours. Cells were lysed in Laemmli buffer/benzonase and the denatured samples applied to a 3-8% Tris-acetate gel and separated by one-dimensional gel electrophoresis. Separated proteins were transferred by electroblotting to a membrane.

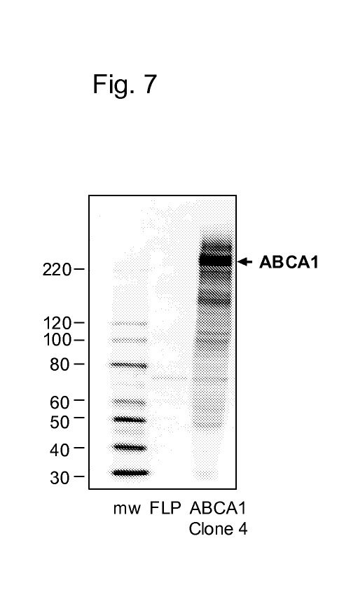

ABCA1 was detected by incubation with a mouse monoclonal Ab ABCA1 (Neuromics) followed by a Goat anti-mouse IgG-HRP (Abcam # 20043) (FIG. 7).

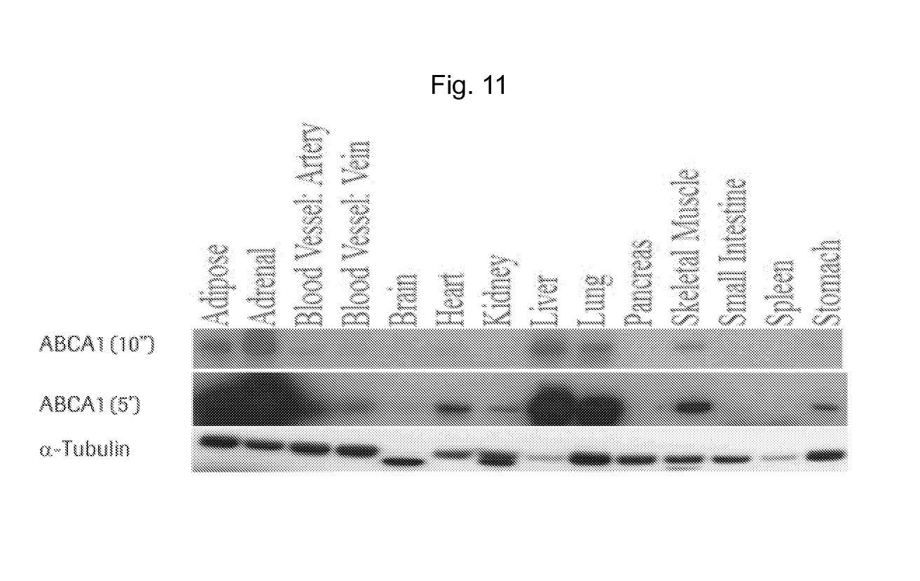

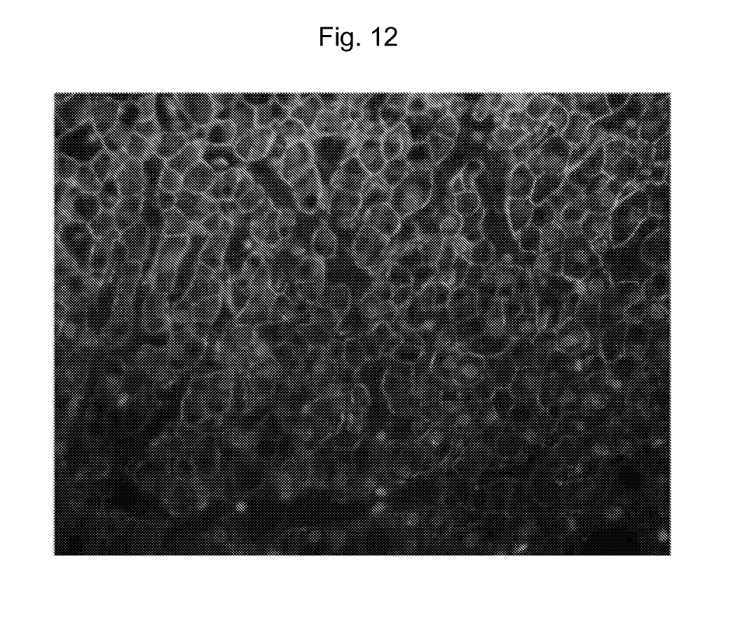

Western blot analysis of human tissue: Human tissue lysates from Biochain Institute, Inc., Hayward, Calif. 94545, USA were used. The blotting was done the same way as described in the previous paragraph. ABCA1 was detected by incubation with mouse anti-ABCA1 mAb (clone 3/84) followed by Goat-anti-mouse IgG-HRP (FIG. 11).

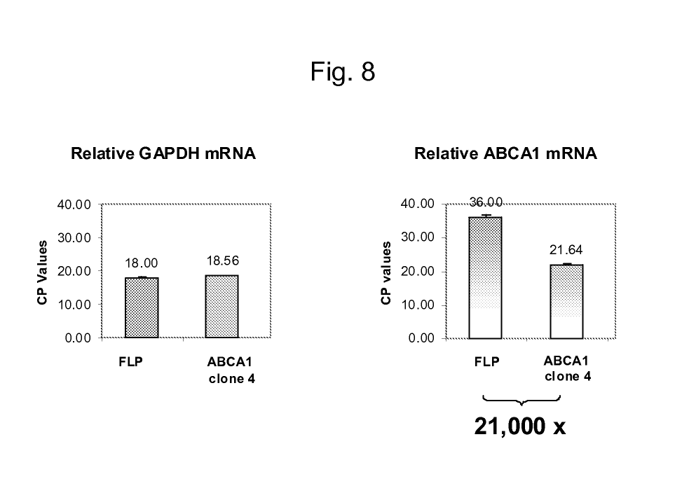

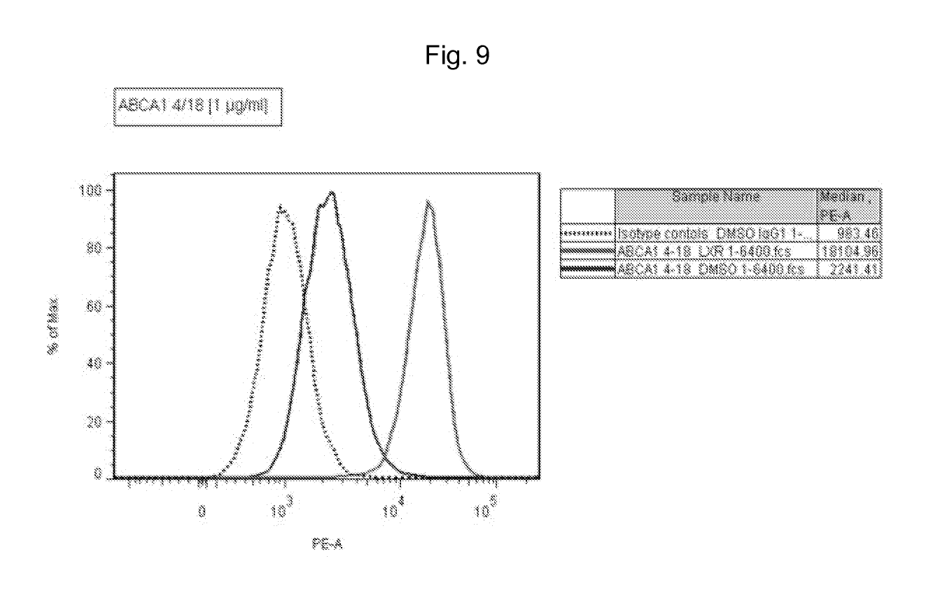

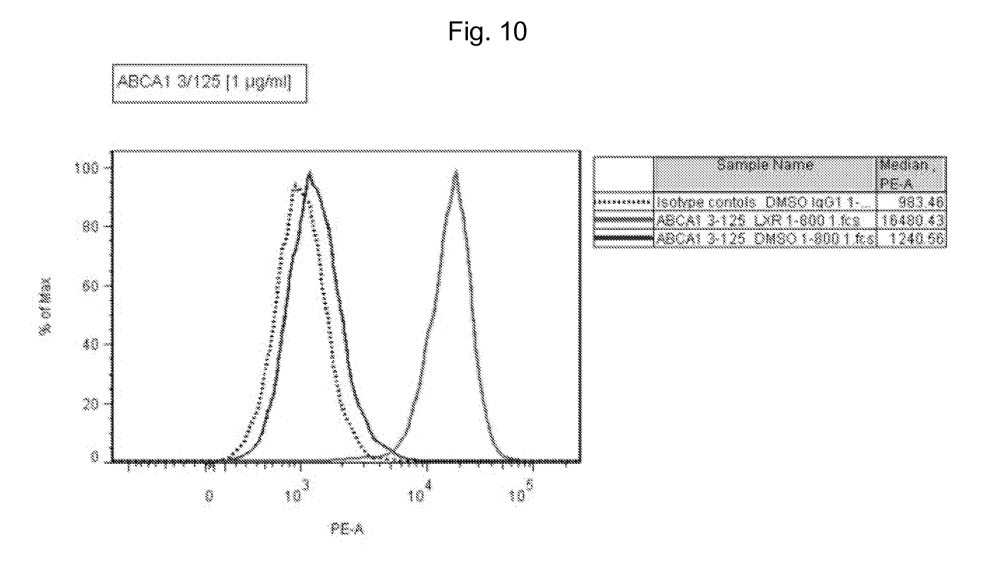

Quantitative PCR analysis of ABCA1 mRNA expression: FLP or Clone4 cells were cultured at 5×10⁵ cells per well in 96 well plates for 48 hrs at 37°. Total RNA was isolated using an automated system according to manufacturer's instructions (Qiagen). Real-time quantitative RT-QPCR was performed on a Lightcycler 480 instrument (Roche) using a one-step reagent mix (Qiagen) and probe/primer sets (Applied Biosystems) to detect relative expression of ABCA1 and GAPDH mRNA's. Data are expressed as the mean Ct values (N=8 wells/condition) +/−SD (FIG. 8).

Antibodies and 2^{d} Step Reagents

Commercially available primary monoclonal antibodies tested for specificity against ABCA1 were from Abcam® and Novus® (clone HJ1 and clone AB.H10). Rabbit polyclonal antibodies tested were from Abcam® (ab81950). Second step reagents Streptavidin PE and goat-α-mouse IgG PE were from Southern Biotechnology®.

In addition to the commercial antibodies, three murine anti-ABCA1 hybridoma supernatants, generated in-house, were evaluated for their performance in detecting ABCA1 in flow cytometry. Detection was performed by applying a secondary reagent (Streptavidin PE, goat-anti-mouse IgG PE and IgG1 PE from Southern Biotechnology®). Hybridoma supernatants of two clones (clone ABCA1-3/84 and ABCA1-3/125) were purified, retested and titrated. At a later time point, clone ABCA1-3/18 derived antibody was tested and compared to ABCA1-3/125 using THP-1 cells. The following antibodies were used for the identification of specific human blood subsets: CD3 Pacific blue, clone UCHT1, CD14 PerCP-Cy5.5, clone M5E2, CD15 APC, clone HI98, CD16 APC-Cy7, clone 3G8, CD19 PE-Cy7, clone SJ25CI and CD66b FITC, clone G10F5. All CD marker specific antibodies were obtained from Becton Dickinson®.

FACS Analysis of FLP 293, FLP 293 Derived Cells and THP-1 Cells

For FACS analysis, FLP 293 cells were rinsed with D-PBS without Calcium and Magnesium (Gibco®) and incubated with 0.02% EDTA (Sigma®). After harvesting, they were washed twice with cold D-PBS before proceeding with the antibody staining Briefly, 0.8-1×10⁶ cells/sample were resuspended in 100 μl BD stain buffer/2% FCS (Becton Dickinson®) containing the 1:200 diluted primary antibody. The incubation time was 45 minutes at +4° C. in the dark. The cells were washed once with cold BD stain buffer/2% FCS and 100 μl second step reagent diluted in BD stain buffer/2% FCS was added. After 45 minutes incubation at +4° C. in the dark and two washes with cold BD stain buffer/2% FCS the cells were analyzed. Non-adherent THP-1 cells were processed in the same way as described above for FLP 293 cells but omitting the incubation in EDTA containing buffer.

Cells were analyzed on a FACS Canto II (Becton Dickinson®). Evaluation was done with FlowJo software (Tree Star®).

ABCA1 Whole Blood Assay and FACS Staining

For whole blood FACS analysis, the anti-ABCA1 clone 3/125 generated in-house was used in all cases. Whole Na-Heparin blood was incubated with the indicated agonists for 24 hours at 37° C. After the incubation, primary anti-ABCA1 specific antibody was added to 100 μl of whole blood at a final dilution of 1:800 for 30 minutes on ice in the dark.

Red blood cells were lysed with lx red cell lysis buffer (Becton Dickinson®) and washed. Subsequently, 100 μl of a secondary goat-a-mouse IgG₁ PE (Southern Biotechnology®) diluted 1:250 was added and incubated for 20 minutes at +4° C. followed by two washing steps.

Cell subset specific antibodies were added for 20 minutes at +4° C. in the dark followed by a washing step before analysis.

Cells were analyzed on a FACS Canto II (Becton Dickinson®). Evaluation was done with FlowJo software (Tree Star®).

Immuno Histochemistry Staining

Human liver FFPE sections (5 mm, Biochain) were dyhydrated, microwaved with citrate buffer (Thermo Scientific), and incubated with mouse anti-ABCA1 mAb (clone 3/84, 1 mg/ml) for 1 hour, followed by the secondary antibody ditection (1 mg/ml for 1 hour, Alexa Fluor® 488 donkey anti-mouse IgG (H+L), Invitrogen, Basel, Switzerland) and DAPI staining

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An isolated antibody that binds to ABCA1 polypeptide, wherein the antibody binds to native ABCA1 polypeptide.

2. The isolated antibody of claim 1, wherein the ABCA1 polypeptide is human ABCA1 polypeptide.

3. The isolated antibody of claim 1, which is a monoclonal antibody.

4. The isolated antibody of claim 1, wherein the antibody has been produced by immunizing suitable animals with whole cells expressing the ABCA1 polypeptide.

5. The isolated antibody of claim 1, wherein the antibody comprises a CDR3 of a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111) and a CDR3 of a VL domain of an antibody obtainable from a hybridoma cell selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

6. The isolated antibody of claim 1, wherein the antibody comprises CDR1 to CDR3 of a VH domain of an antibody obtainable from a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111) and a CDR1 to CDR3 of a VL domain of an antibody obtainable from a hybridoma cell selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

7. The isolated antibody of claim 1, wherein the antibody comprises a VH domain and a VL domain of an antibody obtainable from hybridoma cell line selected from the group consisting of ABCA1-3/84 (DSM ACC3109), hybridoma cell line ABCA1-3/125 (DSM ACC3110) and hybridoma cell line ABCA1-4/18 (DSM ACC3111).

8. The isolated antibody of claim 1, wherein the antibody is produced by hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

9. A hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

10. An isolated nucleic acid comprising a sequence encoding:
(i) a VH domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111),
(ii) a VL domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111), or
(iii) a VH and a VL domain of an antibody obtainable from a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

11. The isolated nucleic acid of claim 10, comprising a sequence encoding an antibody produced by a hybridoma cell line selected from the group consisting of hybridoma cell line ABCA1-3/84 (DSM ACC3109), hybridoma cell line ABCA1-3/125 (DSM ACC3110) and hybridoma cell line ABCA1-4/18 (DSM ACC3111).

12. A vector comprising the nucleic acid of claim 10.

13. A host cell comprising the vector of claim 12.

14. A method of producing an antibody comprising culturing the host cell of claim 13 so that the antibody is produced.

15. A method for the detection of ABCA1 polypeptide in a tissue sample of an animal comprising:
a) providing a tissue sample of the animal,
b) detecting ABCA1 polypeptide in the sample of step a) using an antibody that binds to native ABCA1 polypeptide.

16. The method of claim 15, wherein the tissue sample is whole blood.

17. The method of claim 15, wherein the animal is a human subject.

18. The method of claim 15, wherein detection of ABCA1 polypeptide in step b) is done by Flow Cytometry.


SEQUENCE LISTING (1 sequence)

SEQ ID NO: 1 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV